(19) United States
(12) Patent Application Publication
(10) Pub. No.: US 2006/0041200 A1
(43) Pub. Date: Feb. 23, 2006

(54) Physiological sensor device

(76) Inventors: James E. Dotter, Boulder, CO (US); David J. Farrell, Louisville, CO (US); James A. Gilbert, Boulder, CO (US)

Correspondence Address: MARSH, FISCHMANN & BREYFOGLE LLP, 3151 SOUTH VAUGHN WAY, SUITE 411, AURORA, CO, 80014, US

(21) Appl. No.: 10/862,093

(22) Filed: Jun. 4, 2004

(51) Int. Cl. A61B 5/02

(52) U.S. Cl. 600/502

(57) ABSTRACT

A heart rate monitoring system for use in sport and fitness training that includes a sensor belt worn on the waist of the user and a display device worn on the wrist of the user. The sensor belt has a pair of sensors that can be located on opposite sides of the body in various locations, such as near the kidneys, near the ilium bones in the pelvis, or on the front abdominal area of the user. Various signal processing techniques are employed to extract the degraded signal that is available at the waist area as compared to the chest area. These signal-processing techniques include bandpass filtering, an adaptive time window, a delayed transmission to allow for correction methods such as missed beat replacement and extra beat deletion, a lead off detection and adjustment algorithm, automatically adjustable gain control, and waveform detection and characterization.

The present invention relates generally to a sensor device for measuring heart rate during exercise and displaying this information for the user, and more particularly to an improvement relating to the position of the sensor and to algorithms related thereto.

### BACKGROUND OF THE INVENTION

Equipment for measuring electrical signals associated with the human heart were first developed over 110 years ago. This branch of science, known as electrocardiography, is used to produce a representation of the electrical signals in the heart known as an electrocardiogram (often abbreviated as EKG or ECG). Today, an ECG commonly involves the use of twelve to fifteen electrodes placed at various locations on the body. ECGs are used for a variety of purposes, but generally they are all diagnostic in nature. That is, ECGs are commonly used to diagnose diseases related to the heart. Because of the great importance of this issue and because of the number of electrodes used and the requirement for accurate placement of the electrodes, ECGs are normally only taken by a doctor or specifically-trained nurse or technician.

In more recent times, portable or ambulatory ECG equipment has been developed that allows a doctor to monitor a patient's ECG signal over an extended period of time while the patient is away from the doctor's office, clinic, or hospital. Such devices may sense and transmit ECG signals through a telecommunications link to the doctor or may record the ECG signals in memory associated with the portable equipment for subsequent evaluation by the doctor. Again, these portable devices typically include twelve or more electrodes and they are utilized to diagnose heart disease.

Over the past several decades, there has been an explosion in the interest and the number of devices relating to personal fitness. One such popular device is known as a heart rate monitor, such as those marketed by Polar Electro OY of Finland. Typical heart rate monitors sold by Polar and other manufacturers are relatively simple devices to operate and are designed for use by an individual without assistance from any medical professional and without any training. Such a heart rate monitor typically includes a strap that is fastened around the chest of the user, in the vicinity of the user's heart. The strap includes a pair of sensors, one located on the left side and one on the right side of the user's breastbone or sternum. The signals from the two sensors are passed through a passive filter in the strap and provided as inputs to a differential amplifier also located in the strap. From there, the signals are coupled to a receiver and display device typically worn on the user's wrist in a design that looks much like a wristwatch.

Because the heart's electrical signals originate at a node in the heart, the strength of the signal measured by the sensor will be greater if the sensor is located closer to the heart. Primarily for this reason, sensor straps for heart rate monitors have always been designed to be worn on the user's chest. In fact, the magnitude of the electrical signal drops rapidly as the sensor is positioned further from the user's chest. Nevertheless, manufacturers of heart rate monitors believe that a certain segment of the exercising population has refrained from using heart rate monitors because of discomfort or other issues caused by the sensor strap being worn at the chest location. This discomfort includes problems for some wearers relating to a tendency of the strap to slip down off of their chest toward their lower chest and waist regions. For female users, there is the issue of the strap interacting with the women's bra or other undergarments. In addition, for certain exercisers that are breathing heavily, the strap may be uncomfortable to the extent it constricts the user's chest. Further, to properly position or remove the chest strap, a user may find it necessary to remove their shirt, which may not be desirable in certain situations.

It is against this background and with a desire to improve on the prior art that the present invention has been developed.

### SUMMARY OF THE INVENTION

In view of the foregoing, a broad objective of the present invention is to provide an improved heart rate monitoring device for individuals to use in monitoring their exercise level. Another objective of the present invention is to improve the comfort of users wearing heart rate monitors. Another objective is to provide reliable heart rate data while maintaining this improved level of comfort.

In carrying out these and other objectives, features, and advantages of the present invention, a heart rate monitoring system is provided for use by a wearer in monitoring the wearer's heart rate, the wearer having a body with skin, a waist, and a centerline dividing the body into a right side and a left side. The system includes a belt that fits around and is positioned on the waist of the wearer; a plurality of sensors retained within the belt in a position to be held against the wearer's skin, each sensor sensing the voltage potential of the portion of the skin that sensor is in contact with, there being at least one sensor positioned on the left side and at least one sensor positioned on the right side of the wearer's body; and electronic devices receptive of the sensed voltage potentials from the plurality of sensors and operative to determine a heart rate of the wearer.

The waist of the wearer may include an area on the wearer's body including and extending between a point below the rib cage and a point at or above the hip region. The system may further include a display device worn on the wearer's wrist that displays the determined heart rate. The plurality of sensors may include a pair of sensors, each located on the back of the wearer proximate to a portion of the wearer's kidneys, each located on the sides of the wearer proximate to a portion of the wearer's ilium bones, or each located on the front of the wearer along the wearer's front abdominal area.

The electronic devices may be located within the belt. The system may further include a remote device that is located at a spaced apart position from the wearer that receives the heart rate determined by the electronic devices. The system may further include a wireless communication link between the electronic devices and the remote device. The remote device may include a display device to display the determined heart rate. The remote device may include a data storage device to store the determined heart rate.

Another aspect of the present invention relates to a method of monitoring a heart rate of an individual. The method includes providing a belt having a plurality of sensors thereon which can sense the voltage potential of human skin that is proximate to the sensors; positioning the belt on the waist of the individual in a position so that at least one sensor is positioned on the left side of the individual and at least one sensor is positioned on the right side of the individual; sensing the voltage potentials of the portions of skin proximate to the sensors; and determining the heart rate of the individual from the sensed voltage potentials.

The method may further include displaying the determined heart rate, passing the determined heart rate information to a remote device that is located at a spaced apart position from the wearer, and/or storing the determined heart rate in a data storage device. The determined heart rate may be passed to the remote device via a wireless communication link.

Numerous additional features and advantages of the present invention will become apparent to those skilled in the art upon consideration of the further description that follows.

### BRIEF DESCRIPTION OF THE DRAWINGS

FIG. 1 is a front view of portions of a user's body with the heart rate monitoring system of the present invention located thereon.

FIG. 2 is a front view, similar to that of FIG. 1, showing possible areas on the user's body where the sensors of the heart rate monitoring system could be located.

FIG. 3 is a pair of side views of a user's body, showing additional possible areas on the user's body where the sensors of the heart rate monitoring system could be located.

FIG. 4 is a rear view of a user's body, showing additional possible areas on the user's body where the sensors of the heart rate monitoring system could be located.

FIG. 5 is a block diagram view of portions of the heart rate monitoring system of the present invention, showing the major components in the waist belt and the display device.

FIG. 6 is a block diagram view of portions of the heart rate monitoring system of the present invention, showing the major components in the waist belt and the remote device.

FIG. 7 is a representation of an electrical signal measured on the body representative of signals that drive the heart muscles, with the magnitude of the electrical signal being plotted versus time.

FIG. 8 is a flowchart of logic in a lead off detection algorithm associated with the heart rate monitoring system of the present invention.

### DETAILED DESCRIPTION OF THE PREFERRED EMBODIMENT

Reference will now be made to the accompanying drawings, which assist in illustrating the various pertinent features of the present invention. Although the present invention will now be described primarily in conjunction with a heart rate monitor having a plurality of sensors on a belt, it should be expressly understood that the present invention may be applicable to other applications where it is required/desired to measure electrical signals on the skin of a wearer. In this regard, the following description of a heart rate monitor is presented for purposes of illustration and description. Furthermore, the description is not intended to limit the invention to the form disclosed herein. Consequently, variations and modifications commensurate with the following teachings, and skill and knowledge of the relevant art, are within the scope of the present invention. The embodiments described herein are further intended to explain modes known of practicing the invention and to enable others skilled in the art to utilize the invention in such, or other embodiments and with various modifications required by the particular application(s) or use(s) of the present invention.

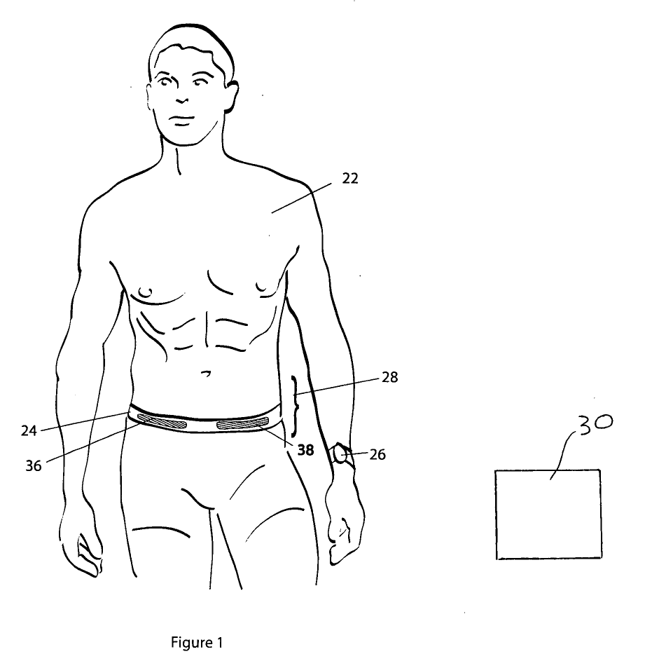

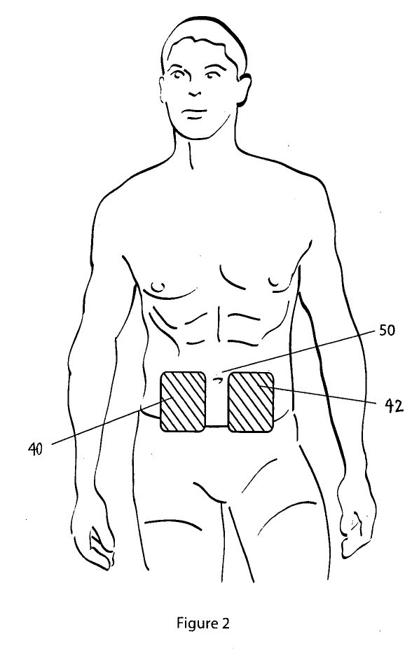

A heart rate monitoring system of the present invention is shown as worn by a user 22 in FIG. 1. The system includes a belt 24 that senses electrical signals on the skin of the user and determines the heart rate of the user therefrom. This heart rate information is then communicated to a display device 26 worn on the wrist of the user 22. Most importantly, the belt 24 is worn in a position on the waist 28 of the user 22. For purposes of this invention, the waist 28 of the user 22 is defined to include a region including and between a point at the bottom of the user's rib cage and the user's hip region. In addition, the heart rate information can be communicated to a remote device 30 not located on the user 22.

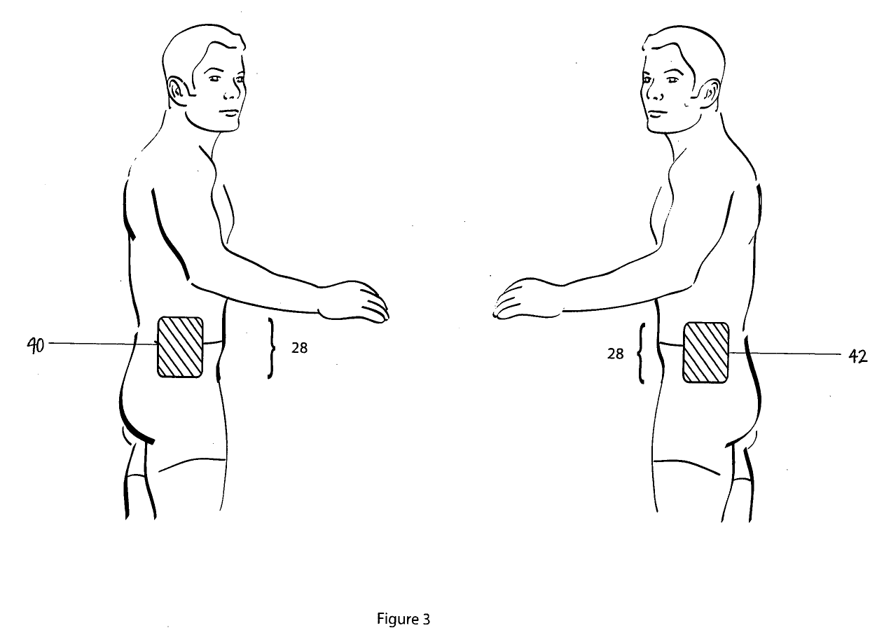

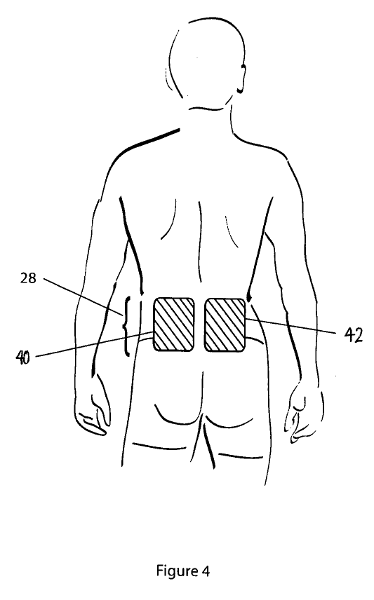

The belt 24 includes a pair of electrical sensors 36 and 38 as shown in FIG. 1. The sensors 36 and 38 may be located on the belt 24 in a position to be placed proximate to an area of the user's body containing the user's kidneys. This is shown in FIG. 4 by areas 40 and 42 on the back of the user's waist. Note that this position would place the sensors 36 and 38 on opposite sides of the user's backbone. Alternatively, those sensors 36 and 38 on the belt 24 may be positioned on opposite sides of the user's waist 28 in areas 40 and 42 so as to be proximate to the ilium bones that are a part of the user's pelvic bones, as shown in FIG. 3. As another alternative, the sensors 36 and 38 on the belt 24 may be positioned to be on the front of the user's waist 28 in areas 40 and 42, on opposite sides of the user's navel 50, as shown in FIG. 4. The sensors 36 and 38 could be composed of any suitable, flexible, conductive material, such as the sensor materials currently used in heart rate monitors marketed to individual users, such as those sold by Polar Electro OY, Nike, and Timex. In some cases, these electrode materials are believed to include a multitude of electrically-conductive spherical beads that are retained in the strap or belt for positioning near the user's skin and having a standard conductive electrode on an opposite side thereof.

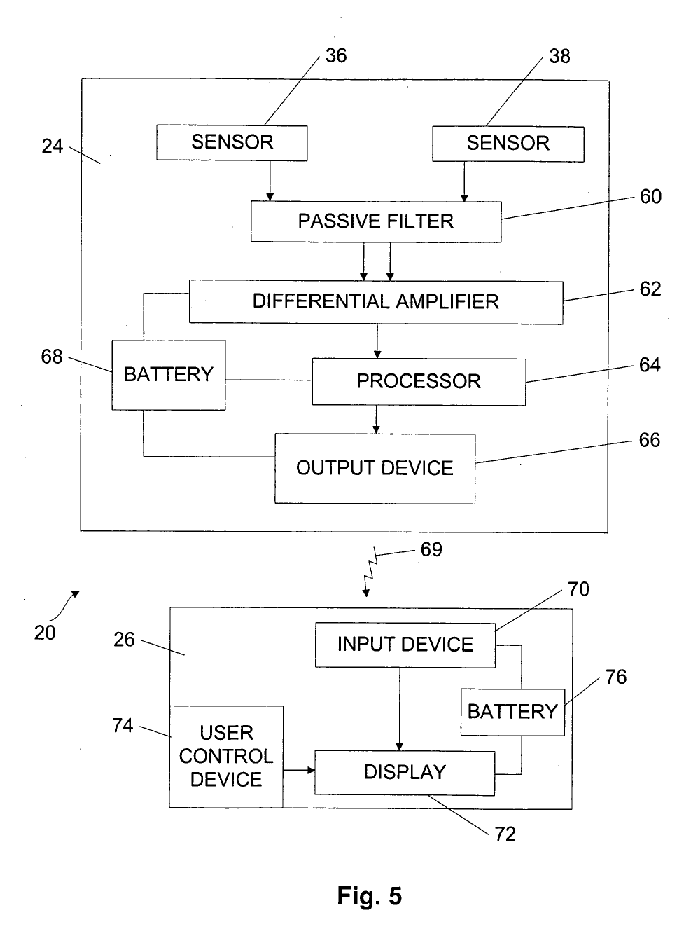

Components of the system are illustrated in block diagram form in FIG. 5. As can be seen, the belt 24 contains the sensors 36 and 38, which provide an electrical signal to a passive filter 60 that provides a filtered output to a differential amplifier 62. The differential amplifier provides an output to a processor 64, which in turn provides a signal to an output device 66. A battery 68 located within the belt 24 powers each of the differential amplifier 62, the processor 64, and the output device 56.

An electromagnetic signal 69 is passed between the output device 66 of the belt 24 and an input device 70 of the display device 26. The input device 70 provides a signal to a display 72 that displays heart rate information on the display device 26. The display device 26 also includes a user control device 74 that provides signals to the display 72 to select/control operational modes and what type of information is displayed on the display 72. A battery 76 provides power to the input device 70, the display 72, and optionally to the user control devices 74.

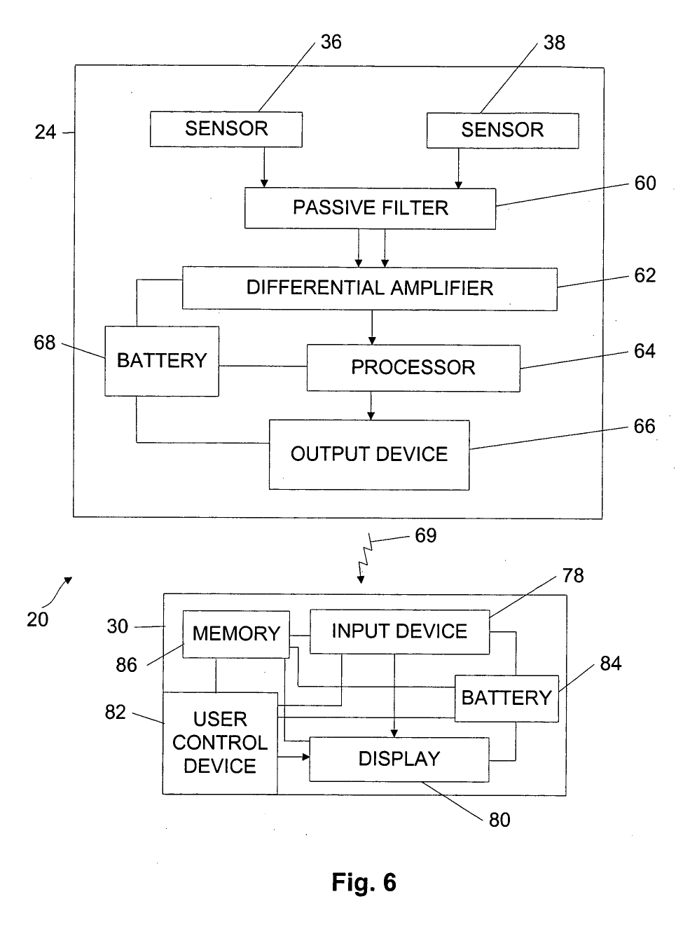

Instead of or in addition to the display device 26 worn on the wrist of the user 22, the electromagnetic signal could be passed to the remote device 30 (FIGS. 1 and 6) which may include other equipment such as a receiver and which may be stationed adjacent to or as part of a treadmill or other stationary exercise device such as a bicycle trainer, a running track, playing field, tennis or basketball court, or the like. As shown in FIG. 6, the remote device can include an input device 78 which may be similar to the input device 70, a display 80, a user control device 82, a battery 84 (or a connection to an AC power source), and a data storage device or memory 86. The data storage device 86 may include any suitable form of memory including magnetic, electronic, or optical, or combinations thereof, including compact flash memory, smart cards, memory ICs, and so forth. As another alternative, the information relating to the heart rate could be stored in the belt 24 for later download to other equipment.

The output device 66 of the belt 24 and the input device 70 of the display device 26 (or the input device 78 of the remote device 30) may communicate via an RF transmitter, receiver, and associated antennas or they may communicate by magnetically coupling together the two circuits via a relatively low frequency magnetic field in the range of 5 kHz. In the latter case, large coils could be used to provide the magnetic coupling.

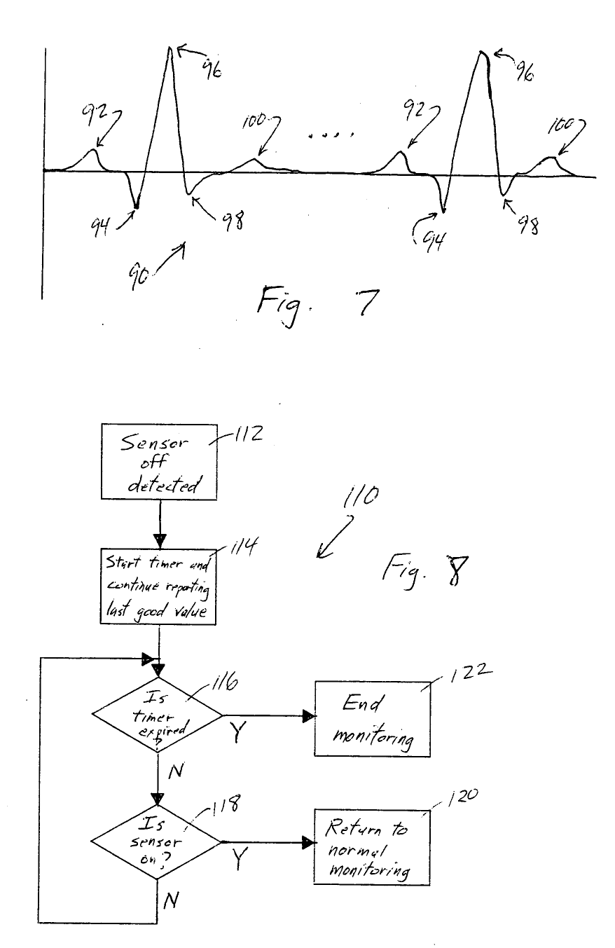

There are other electrical signals in/on the human body that are essentially noise sources in determining heart rate. These include electrical signals sent to other parts of the body to cause muscular activity such as movement of the arms and legs such as will occur during exercise. It is desirable to be able to ignore those other electrical signals and focus on those electrical signals from the heart. A representation of the electrical signal from the heart is shown in FIG. 7. An electrical signal 90 is shown plotted versus time. The electrical signal 90 includes several characteristic features that are repeated on a periodic basis. These features have been given standard names in the science of electrocardiography. For example, one portion of the signal is known as the P wave 92. A second portion of the signal is known as the Q wave 94. The Q wave 94 is followed by the R wave 96, which is followed by the S wave 98 and the T wave 100. Sometimes the combination of the Q wave 94, R wave 96, and S wave 98 are referred to as the QRS complex. In FIG. 7, some elapsed time is represented between the first series of the P wave 92 through T wave 100 and a second series of a P wave 92 through T wave 100. It is the amount of total elapsed time between identical portions of the electrical signal that can be used to determine the heart rate. For example, the elapsed time from one R wave 96 to the next succeeding R wave 96 is known as the inter-beat timing or R-to-R variability.

The QRS complex of the electrical heart signal typically occurs in approximately 65 milliseconds, which corresponds to a frequency of approximately 15 Hz. Bandpass filtering can be used to remove electrical signals significantly above or below this 15 Hz range and this can eliminate a good deal of the noise. Either the passive filter 60 or the processor 64 could perform this bandpass filtering. Signals that are filtered out include those related to motion artifact, power line inductive or capacitive coupling, DC or baseline electronics wander, muscular activities, thermal noise and galvanic action noise from two dissimilar materials in contact. This first noise source is low frequency noise as a result of the imperfect connection between the skin and the electrode sensors, while the characteristics of the last noise source depend upon the material choices and construction of the electrode. It should be understood that while the noise sources are not completely characterized, at a minimum there are internal sources of noise such as signals internal to the human body, there are external sources of noise such as fluorescent lights or other electrical equipment operating nearby, and there is circuit noise associated with the heart rate monitoring system itself.

While the P wave 92 through T wave 100 shown in FIG. 7 may be representative of an electrical signal if sufficient resolution is available, signals measured at the waist level have a very different signature, with the QRS complex possibly being the only detectable element, and it may take a different shape. It has also been discovered that the time period of this QRS complex is relatively constant for a given individual at different heart rates and is relatively constant from individual to individual.

It has been determined that the amount by which the inter-beat timing can vary from heartbeat to heartbeat is finite. That is, with a normal, healthy human heart, beating at a particular rate, it can be determined that the next heartbeat will not happen sooner than a particular time after the last beat or before a certain longer time after the last beat. In other words, it is possible to define a window during which the next heartbeat should occur. Of course, the opening and closing of the window around the next anticipated heartbeat is not necessarily symmetrical because it has been determined that the heart rate can speed up quicker than it can slow down. It is believed that the use of such a time window in which to look for the next heartbeat is novel since there is little reason to have diagnostic ECG equipment use a window and exclude data not in the window. Furthermore, earlier heart rate monitors used for exercise never previously used this type of signal processing technique, in part because the electrical signal is so strong with the strap located along the chest. Furthermore, the time window can be adaptive in that the size of the window is increased or decreased depending on the measured heart rate. In this manner, certain noise and spurious signals can be ignored. One source of these spurious signals may be interruptions and interference related to the environment, ambience (e.g., nearby power lines, automobiles, other athletes, and the like), and actions of the user. It is also possible to use a rolling average of the last X beats to determine the heart rate, and thus determine the size of the adaptive time window. Of course, since using a rolling average is a means of filtering, there will be some adjustment to make the window larger just due to the larger number of beats that the heart rate is filtered across.

Another signal processing technique that has been determined to be useful in the heart rate monitoring system of the present invention is delaying transmission of the measured heart rate to allow for correction methods. This allows an algorithm in the microprocessor 64 to look at a series of actual beats and determine, based on a rolling average, whether heartbeats have been missed which should be inserted, or whether erroneous heartbeats have been detected which should be removed. Once these error corrections have been made, then transmission to the display device 26 (or remote device 30) can occur. Of course, there is a delay in the reporting of the data so the data being displayed is somewhat stale, but it is believed that this particular disadvantage is more than offset by obtaining a more accurate heart rate. The rolling average, together with the known maximum rate of change of the inter-beat timing, can be used to determine when a beat has been missed or an erroneous beat has been detected.

It has also been determined that another signal processing technique is desirable as part of the heart rate monitoring system of the present invention. A circuit is devised that can raise an alarm when one of the sensors 36 and 38 touching the skin is removed from the skin. This may be a temporary dislocation, or it may be a more permanent dislocation in which the user has removed the waist belt 24. In the case of a temporary dislocation, it is desirable to continue monitoring the heart, and to infer the behavior of the heart during the time that the sensor is off of the skin, based on recent history. An algorithm is devised to continue reporting inferred heartbeats for a period of time, in anticipation that the sensor(s) will be replaced and heart rate monitoring will be continued. This eliminates momentary disruptions of heart rate reporting to the user, and maintains a more realistic record of heart rate activity for later review. The detection of the sensor being off the skin is performed by detecting the impedance of each sensor relative to the other sensor or to circuit ground. With a DC current, this may be approximately 1 kΩ or with an AC current, this may be in the range of 50 Ω. If this impedance is greatly exceeded, the system can realize that the sensor is off the skin.

The flowchart of FIG. 8 shows the logic performed by the algorithm 110. The algorithm 110 is only performed when one of the sensors is detected (112) to be off of the user's skin. Next, a timer is started (114) and the last good heart rate value continues to be reported. Then, a test is performed (116) to see if the timer has expired. If the timer has not expired, then another test is performed (118) to see if the sensor is back on the skin. If the answer is yes, then the algorithm returns (120) to normal monitoring. If the answer to the test performed (118) is no, then the processing returns to the test (116) which determines if the timer has expired. If the timer has expired, then monitoring is ended (122).

Another signal processing technique that has been discovered to be useful with the heart rate monitoring system of the present invention is automatically adjustable gain. It has been discovered that the strength of the electrical signal varies greatly depending on the location on the user's body and that the strength of the electrical signal varies greatly from user to user. The adjustable gain circuit initially searches for a usable electrical heart rate signal across a wide range of signal levels. Once a signal is identified, the detecting circuit can be fine tuned to the specific signal strength in an adaptive manner in order to provide a more reliable reporting of the heart rate.

It has also been discovered that certain signal processing techniques are useful to account for the fact that the heart rate monitoring system will not be operated by trained professionals. For example, it is possible that a user will place the belt 24 in an upside-down position or in locations not intended. In the case of the belt being worn upside-down, the result may be that the electrical signal is inverted so that the P wave is a negative signal. A circuit and algorithm can be used for identifying the shape of the electrical signal and informing other parts of the electronics (e.g., the differential amplifier 62) of the inverted nature of the signal, so that necessary adjustments can be made.

Prior art chest strap equipment does not work on the waist area of a user's body because the signal levels measurable at the waist area are so low/degraded. Thus, the signal-to-noise ratio is low, since the noise sources generally stay the same while the signal level drops significantly. The electrical signal available at the waist appears to be approximately ten times smaller in magnitude than the electrical signal available at the chest.

The optimal position for a heart rate monitoring system would be where the signal is strongest, on the chest. For reasons other than signal quality, the inventors have been motivated to place the sensors at the waist portion. It is believed that no one else has previously been motivated to measure heart rate at this sub-optimal position. In fact, it is believed that all prior art taught away from placing the sensors at any sub-optimal position.

One possible scenario for use of the heart rate monitoring system of the present invention is that a user could wear the belt 24 on their waist for certain exercises and on their chest for other exercises. The fact that certain signal processing techniques have been used to extract a degraded signal at the waist location does not prevent operation of the system on an area of the chest where the signal is not so degraded.

It is believed that it is desirable to have the sensors on opposite sides of a plane running longitudinally through the user's body parallel to the spine and intersecting the navel (dividing the body into left and right sides corresponding to one's left and right hand) because of the nature of the electrical signals conducted through the body. It is generally believed that the wavefront of the signal on one side of the body emanating from a node in the heart is out of phase with the wavefront of the signal on the other side of the body emanating in an opposite direction from the heart node. For this reason, it is believed that little to no signal would be available if those sensors were placed on the same side of the plane or centerline while a stronger signal can be obtained if the measurement is made from opposite sides of the centerline.

It is believed that the heart rate monitoring system of the present invention would work suitably with sensors placed nearly anywhere on the body as long as there is one on each of the two sides of the body. In the waist region, this could include any combination of one sensor anywhere on the waist on the left side of the body and a second sensor anywhere on the waist on the right side of the body.

It may be that sensors on the front of the waist are not as effective as those on the side or back of the waist, particularly if the user is overweight with a large belly that may cause intermittent disruption of the signal because the sensors are bounced around too much. There may also be a wide variability in signal strength at the waist due to sweat, which is a good conductor.

A high dynamic range, high resolution (e.g., 24-bit) analog-to-digital converter (ADC) can be useful, because it reduces the signal processing and adaptive gain requirements of the front end. More can be done algorithmically (digitally) within the processor or an integrated circuit.

There are several advantages of the heart rate monitoring system of the present invention. The advantages primarily relate to the location of the sensor strap around the waist portion of the user's body. This position provides comfort, convenience, and aesthetic improvements. The familiarity of a waist belt as opposed to a chest strap is also a benefit to many users. The advantages also include enhanced breathability of the wearer/athlete (especially during maximal effort), reducing chafing (especially relative to arm movement during running), and a more natural and familiar location for a constricting device which will probably be indistinguishable from the elastic in the athlete's shorts or pants.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain best modes known of practicing the invention and to enable others skilled in the art to utilize the invention in such, or other embodiments and with various modifications required by the particular application(s) or use(s) of the present invention. It is intended that the appended claims be construed to include alternative embodiments to the extent permitted by the prior art.

1. A heart rate monitoring system for use by a wearer in monitoring the wearer's heart rate, the wearer having a body with skin, a waist, a chest region, and a centerline dividing the body into a right side and a left side, the system comprising:
a belt that fits around and is adapted to be positioned on the waist of the wearer;
a plurality of sensors retained within the belt in a position adapted to be held against the wearer's skin, each sensor sensing the voltage potential of the portion of the skin that sensor is in contact with, there being at least one sensor adapted to be positioned on the left side and at least one sensor adapted to be positioned on the right side of the wearer's body wherein the sensors are each positioned such that a straight line drawn through the body between any two of the sensors does not run through the chest region; and
electronic devices receptive of the sensed voltage potentials from the plurality of sensors and operative to determine a heart rate of the wearer.

2. A heart rate monitoring system as defined in claim 1, wherein the waist of the wearer includes an area on the wearer's body including and extending between a point below the rib cage and a point at or above the hip region.

3. A heart rate monitoring system as defined in claim 1, further including a display device adapted to be worn on the wearer's wrist that displays the determined heart rate.

4. A heart rate monitoring system as defined in claim 1, wherein the plurality of sensors include a pair of sensors, each adapted to be located on the back of the wearer.

5. A heart rate monitoring system as defined in claim 4, wherein the sensors are adapted to be located proximate to a portion of the wearer's kidneys.

6. A heart rate monitoring system as defined in claim 1, wherein the plurality of sensors include a pair of sensors, each adapted to be located on the sides of the wearer.

7. A heart rate monitoring system as defined in claim 6, wherein the sensors are adapted to be located proximate to a portion of the wearer's ilium bones.

8. A heart rate monitoring system as defined in claim 1, wherein the plurality of sensors include a pair of sensors, each adapted to be located on the front of the wearer.

9. A heart rate monitoring system as defined in claim 8, wherein the sensors are adapted to be located along the wearer's front abdominal area.

10. A heart rate monitoring system as defined in claim 1, wherein the electronic devices are located within the belt.

11. A heart rate monitoring system as defined in claim 1, further including a remote device that is located at a spaced apart position from the wearer that receives the heart rate determined by the electronic devices.

12. A heart rate monitoring system as defined in claim 11, further including a wireless communication link between the electronic devices and the remote device.

13. A heart rate monitoring system as defined in claim 11, wherein the remote device includes a display device to display the determined heart rate.

14. A heart rate monitoring system as defined in claim 11, wherein the remote device includes a data storage device to store the determined heart rate.

15. A method of monitoring a heart rate of an individual, the method comprising:
providing a belt having a plurality of sensors thereon which can sense the voltage potential of human skin that is proximate to the sensors;
positioning the belt on the waist of the individual in a position so that at least one sensor is positioned on the left side of the individual and at least one sensor is positioned on the right side of the individual;
sensing the voltage potentials of the portions of skin proximate to the sensors; and
determining the heart rate of the individual from the sensed voltage potentials.

16. A method as defined in claim 15, wherein the waist of the individual includes an area on the individual's body including and extending between a point below the rib cage and a point at or above the hip region.

17. A method as defined in claim 15, further including displaying the determined heart rate.

18. A method as defined in claim 15, further including passing the determined heart rate information to a remote device that is located at a spaced apart position from the wearer.

19. A method as defined in claim 18, further including storing the determined heart rate in a data storage device.

20. A method as defined in claim 18, wherein the determined heart rate is passed to the remote device via a wireless communication link.
